(19) United States
(12) Patent Application Publication
(10) Pub. No.: US 2011/0229424 A1
(43) Pub. Date: Sep. 22, 2011

(54) FORMULA FOR REMOVING COLOR COATS AND VARIOUS SOIL LAYERS FROM SURFACES, METHOD FOR PRODUCING THE AGENT, AND METHOD FOR CLEANING

(75) Inventors: Dirk Schumann, Leipzig (DE); Ralner Surkow, Leipzig (DE)

(73) Assignee: BUBBLES & BEYOND GMBH

(21) Appl. No.: 12/678,809

(22) PCT Filed: Sep. 19, 2008
(86) PCT No.: PCT/EP08/62573

§ 371 Date: Jun. 3, 2011

(51) Int. Cl. A61K 8/92; C11D 3/60; A61Q 5/08; C11D 17/00; B08B 3/00; A61K 8/18

(52) U.S. Cl. 424/62; 510/469; 510/497; 510/496; 510/500; 510/491; 510/109; 510/118; 510/488; 510/417; 134/26; 132/202

(57) ABSTRACT

The invention relates to a cleaning agent, characterized in that the cleaning agent comprises a microemulsion or a fluid nanophase system, and has the following components: a) at least one non-water-soluble substance having a solubility in water of less than 4 g per liter; b) at least one amphiphile substance, NP-MCA, that has no tenside structure, does not form structures on its own, has a solubility in water or oil between 4 g and 1000 g per liter, and preferably does not accumulate at the oil-water boundary, with the provision that NP-MCA is not selected from among 2-Ethyl-1,3-Hexanediol, 2-Methyl-2,4-Pentanediol, 2-(n-Butyl)-2-Ethyl-1,3-Propanediol and/or 1,2-Diols; c) at least one anionic, cationic, amphoteric and/or non-ionic tenside; d) water and/or a water-soluble solvent having hydroxyl functionality and, optionally, additives.

The present invention describes novel cleaning compositions, a process for the preparation thereof and a method for cleaning various surfaces, specifically if these have been treated in a desirable or undesirable manner with dirt, lacquers and paints, colour sprays, lacquer or felt pens or other coloured surface coatings. The cleaning composition according to the invention serves in particular for removal of graffiti from smooth and/or porous substrates, for stripping lacquers from metal, wood or glass surfaces, for removal of dirt, for removal of cosmetics, for decolouring hair and for removal of nail varnish. In this context, the cleaning action of the compositions according to the invention consists of fragmentation of the dirt or colour layers and detachment thereof from the substrate, smearing largely being avoided. This is made possible by the composition according to the invention of specifically extended microemulsions.

At the start of the 1970s spraying and painting of walls and other surfaces spread from New York in the wake of the hip-hop culture. According to an estimate of the German convention of municipal authorities (April 2002), the damage to the economy by graffiti in Germany amounts to about 200 million euros, plus about 50 million in the underground and suburban transport systems and the German railway. Although in many cases well thought-out graffiti (so-called “high graffiti” or “masterpieces”) are an art form, the significant majority are scrawlings (so-called “tags”) and therefore damage to property. The graffiti removers obtainable commercially to date help to only a limited extent or not at all. They are usually effective only on smooth areas and require a long action time of as a rule 30 minutes to 2 hours. In certain cases an action time of up to 24 hours and subsequent treatment with a high pressure cleaner with 90° C. hot water under a pressure of 150 bar is even recommended.

The requirements imposed on a graffiti remover are very high, since the composition must act on different substrates and furthermore graffiti paints are very varied in their composition. Graffiti paints and coatings do not have a uniform composition, i.e. the binders vary according to the manufacturing company, the intended use of the paint and even within a series of products. As a rule, more detailed information on the constituents of their colour products are not divulged by the manufacturing companies.

The sprayed or painted substrates are usually made of concrete, plaster, brick and clinker, but also of fine ceramics and natural stones, such as marble or sandstone. On porous substrates, the paints penetrate into the pores and masonry joints, so that removal is possible only with difficulty. Smooth surfaces, such as lacquered or non-lacquered metals, plastics or glass, are easier to clean.

In addition to the substrate, the coloured pigments also decide the outlay on colour removal. On the one hand, on light-coloured walls dark colour shades leave behind a colour shadow which is already striking in purely visual respects, and on the other hand finer pigment particles are carried into the pores more easily, and then remain in them. In some cases, colour shadows are also generated by pigments with an inadequate “fastness”. In this case the pigments are not completely insoluble, so that pigment molecules for example migrate into a lacquered substrate or through a new coat of paint and lead inter alia to so-called “bleeding”.

The finely divided pigments include above all the carbon blacks (black) and phthalocyanines (green to blue) and some other organic pigments. Among organic pigments of simple structure there are also often those which tend towards bleeding. As a rule, the white, yellow and red mineral-based pigments (titanium dioxide, iron oxides etc.), ultramarine and metallic pigments (aluminium, bronzes) have larger particle diameters and therefore can be dissolved out of the binder more easily.

The binders employed in graffiti paints can be alkyd resins of varying structure (e.g. polyester or fatty acid nature and content), resins, modified or wholly synthetic rubbers, cellulose esters, aldehyde condensates or many other polymers. Solvent-free, water-miscible spray paints are often acrylic or polyvinyl acetate dispersions, and sometimes also acrylic resins dissolved under alkaline conditions.

Removal of graffiti is a specific case of lacquer removal and stripping. The object of removal of lacquer while at the same time protecting the substrate is the same in both cases. In contrast to the removal of graffiti, however, the removal of lacquer or stripping is associated with less difficult, i.e. porous or highly structured, substrates, but in return it is often necessary here to remove crosslinked lacquer systems or coalesced particles of high polymers from emulsion paints, which are distinguished by an insolubility in solvents and a particularly good adhesion to the substrate.

For decades formulations having a universal action, namely powerful solvents, above all methylene chloride, have been used for removal of lacquer. Because of the toxicity of this chlorinated hydrocarbon and an increased environmental awareness of society, these solvents and related hydrocarbons nowadays can no longer be employed without reservation. Thus, in Germany the Technische Regeln für Gefahrstoffe [Technical Regulations for Hazardous Substances] TRGS 6123 are concerned in detail with possible substitutes and methods for methylene chloride. Alternative halogen-free stripping formulations often have the disadvantage that they require relatively long action times, in some cases are highly flammable and do not show an equally good detachment on every lacquer system. Precisely the latter problem manifests itself more often nowadays, since ever more water-based lacquer formulations are coming on to the market, which expect completely different solubilization properties to the solvent-based lacquer systems used to date. For the present large number of lacquer systems, formulations for removal of lacquer which have both hydrophilic and hydrophobic solution properties are thus required. Removal of lacquer moreover concerns not only the detachment of dried lacquer layers, but also the removal of fresh lacquer formulations such as occur in wide fields of use, from a painter's handiwork to the printing inks industry.

A specific field of removal of lacquer is the removal of nail varnish. During removal of cosmetic nail varnishes, in by far the most cases de-oiling and leaching out of the nails and of the surrounding areas of skin occur as a result of the action of the usual organic solvents. On long-term use of nail varnish remover, this manifests itself in the form of a whitening of the nails and skin up to brittle nails without shine. For many decades various proposals have therefore already been made to avoid or even prevent these effects. Although it is known that the de-oiling and the withdrawal of important structural substances from the nail takes place due to the solvents, acetone, ethyl acetate, butyl acetate and the like have been and still are employed in nail varnish removers.

To remove graffiti, in addition to mechanical methods, such as sandblasting, blasting with dry ice or laser ablation, usually liquid or gelatinous graffiti removers are used in the prior art. The advantage of the graffiti removers is the possibility of quick application and the simultaneous action which then starts on the detachment of the paint over relatively large areas, while with the mechanical methods the paint can be detached only locally. The graffiti removers to date contain individual solvents or solvent mixtures, which have the task of physically dissolving the binder of the graffiti lacquer. In some graffiti removers, the solvents are emulsified in water. In this context, the often thinly liquid solvents or emulsions are usually thickened by means of additives to prevent running off on vertical surfaces.

The following solvents, all of which are to be found in the list of the Technische Regeln für Gefahrstoffe [Technical Regulations for Hazardous Substances] (TRGS 612, March 2002), are typical for conventional graffiti removers. They are often esters, such as e.g. shorter and/or longer fatty acid esters, such as e.g. methyl oleate, or fatty acid mixtures, such as rape oil fatty acid methyl ester (“biodiesel”), and various dicarboxylic acid esters, e.g. so-called dibase esters (DBE esters). EP 1772 496 A1 uses such methyl esters, and also cyclohexanone, in some cases in (micro) emulsion for dissolving graffiti paints. Lactic acid esters are also employed now and again. The use of glycol derivatives, such as propylene carbonate, 1-methoxy-2-propanol or 2-methoxy-1-methylethyl acetate, is also widespread.

The ethyl 3-ethoxypropionate described in the Offenlegungsschrift DE 10 2004 015 092 A1 is also structurally similar here. However, ethyl 3-ethoxypropionate has also already been used previously many years ago in DE 691 28 887 T2 (EP 0 551 378 B1) for activation of the strongly polar aprotic solvent 1-methyl-2-pyrrolidone (NMP) and gamma-butyrolactone. Precisely 1-methyl-2-pyrrolidone is a known and excellent lacquer solvent, which is employed either in the pure form or in mixtures with other solvents in many stripping formulations and graffiti removers. DE 695 21 333 T1 thus describes the use of 1-methyl-2-pyrrolidone, but also other pyrrolidones, in amounts of from 1 to 60 wt. % in the formulation. Tetrahydrofuran, which is structurally close to gamma-butyrolactone, is also used in graffiti removers. To replace 1-methyl-2-pyrrolidone, the Offenlegungsschrift DE 10 2004 012 751 A1 describes 1-ethyl-2-pyrrolidone, which was also already a constituent of DE 695 21 333 T1. The N-methylcaprolactam from the Offenlegungsschrift DE 102004015182 A1 can also be regarded as structurally very similar.

Methylene chloride and gamma-butyrolactone are to be avoided because their action is unacceptable to the environment or to health. However, 1-methyl-2-pyrrolidone has also recently been suspected of having a teratogenic action. Many graffiti removers and stripping formulations contain often relatively large amounts of NMP, since it is outstandingly suitable for lacquer removal and up until a short time ago was regarded as not particularly toxicologically unacceptable. Regardless of whether the suspicious factors will bear out in future, its complete replacement also makes sense from the point of view of product marketing.

Short-chain solvent molecules often penetrate rapidly into the binders of graffiti paints, but in most cases also evaporate again rapidly. Long-chain solvent molecules often require considerably more time (e.g. between about 20 minutes to 2 hours) for this operation and lead to swelling of the coatings, so that these can then be rubbed off mechanically from the substrate more easily.

A disadvantage of individual solvents and even of solvent mixtures is that because of the complexity of the dissolving operation, which to date is not yet completely understood in its details, especially with respect to the dissolving of lacquers or polymers, not all binder types can be dissolved equally well. This manifests itself when an attempt is made to dissolve various graffiti paints with the same remover system. Out of a wide range of various aerosol paints, always some of them cannot be satisfactorily detached from the substrate.

Even in the case of a successful dissolving operation, there is a further disadvantage of solvents in that the binder of the graffiti paint is highly diluted and, together with the coloured pigments, is distributed beyond the original edge of the graffito. If the substrate is not smooth but has a structured surface, in many cases residues of the binder and above all the coloured pigments remain in very fine cracks and pores. This manifests itself later in the form of a coloured “shadow”, which can be removed only with great difficulty to not at all. It is to be noted in detail that in this context the substrate does not have to be porous and absorbent at all, like most plasters on the walls of houses, and this shadow formation can also be observed on glazed rough tiles, ceramics or clinkers.

Since the pigment binder composition of graffiti paints to be removed cannot be ascertained at the site of the damage, a graffiti remover must have the broadest possible spectrum of dissolving power. As noted above, solvent mixtures also cannot completely achieve this. A system must therefore be found with which a large number of different binders can be detached from the substrate. The system should be able to superficially dissolve the graffiti paints in a short time, e.g. within 10 minutes, a maximum of up to half an hour, and remove them from the substrate as far as possible without colour shadows. The graffiti remover should contain no substances which are toxic or present long-term health or environmental problems, namely no organic halogen compounds (such as methylene chloride), no gamma-butyrolactone and as far as possible also no N-alkyl-pyrrolidones, but also no strong acids or bases.

Like graffiti removers, stripping formulations should contain no substances which are toxic or present long-term health or environmental problems, namely no organic halogen compounds (such as methylene chloride), no gamma-butyrolactone and as far as possible also no N-alkyl-pyrrolidones. Strong acids or bases should likewise be avoided. The wide applicability and the fast action of stripping formulations containing methylene chloride are still advantageous. However, compositions which are free from methylene chloride often require significantly longer action times, in some cases up to more than 24 hours. Although another greater deep action is thereby achieved, such long periods of time are unacceptable. In addition, the problem of smearing due to the use of solvents or mixtures thereof remains. A suitable stripping formulation therefore should not be a danger to the environment or health, that is to say should contain none of the abovementioned solvents, acids or bases, should act on a very broad range of paints and binders in the shortest possible time, should not cause smearing of the old lacquers, should not attack the various substrates and as far as possible should be removable with water.

As in the case of graffiti removal and removal of lacquer, for removal of nail varnish—but here especially, due to the cosmetic use—the choice of constituents which are the most effective as possible on the one hand and the most environment- and health-friendly as possible on the other hand is necessary. The use of solvents such as methylene chloride (DE 1089515) or carbon tetrachloride (DE 830094) or similarly halogenated hydrocarbons is inconceivable in present-day formulations. However, since recently, new knowledge has also existed of the adverse effects on health of solvents used in nail varnish removers for a long time, such as for example gamma-butyrolactone (GBL).

The modern consumer meanwhile expects of a nail varnish remover not only the functions of cleaning and care of nails and areas of skin, but also acceptable and sensorially discreet constituents. Precisely in recent years there has been an increasing sensitizing of consumers to the pungent or piquant smells of solvents in a large number of products. This applies in particular to the solvents used most frequently in nail varnish removers, such as acetone, ethyl acetate, butyl acetate, ethyl lactate etc.

Although these solvents have proved themselves in nail varnish removers due to their fast activity and relatively low toxicity, their replacement is nevertheless urged on the basis of the characteristic smells and the still existing problems of de-oiling of the skin and nails. The bio-ethanol meanwhile contained in so-called environment-friendly products in turn is not a suitable alternative as a solvent, since only specific nail varnishes can be removed with this, so that a completely novel action system must be found which meets all the abovementioned demands.

A nail varnish remover should thus be capable of removing the applied coatings from the nail rapidly and cleanly, without causing the abovementioned patterns of damage. Highly de-oiling solvents should not be employed or should be employed only in small amounts, and re-oiling and moisturizing substances should be available in a sufficient amount, but must not impede or delay the removal of the varnish. As in the case of aerosol paints and other coloured compositions with which graffiti are produced, there exist a number of polymeric lacquer binders which are used in nail varnish formulations. In addition to nail care, a suitable nail varnish remover must also be capable of detaching all these various varnish compositions equally well from the substrate. As in the case of the graffiti remover, but in this field of use of somewhat lesser importance, the coloured composition should not be smeared when the nail varnish remover is employed.

The object of the present invention was thus to provide a cleaning composition for a wide range of structural substrates and also for skin, hair and finger nails which ensures rapid and thorough removal of the paint or of the lacquer without smearing. The cleaning composition should not be a danger to the environment or health and should dissolve a wide range of paints and lacquers.

The object of the invention is achieved by provision of a cleaning composition, characterized in that the cleaning composition contains a microemulsion or a fluid nanophase system and comprises the following constituents:
a) at least one water-insoluble substance (oil) with a solubility in water of less than 4 g per litre;
b) at least one amphiphilic substance, NP-MCA, which has no aligned hydrophilic-hydrophobic surfactant structure, is not structure-forming by itself, i.e. does not form micelles, the solubility of which in water or oil is between 4 g and 1,000 g per litre and which does not accumulate preferentially at the oil-water interface, with the proviso that the NP-MCA is not chosen from 2-ethyl-1,3-hexanediol, 2-methyl-2,4-pentanediol, 2-(n-butyl)-2-ethyl-1,3-propanediol and/or from 1,2-diols;
c) at least one anionic, cationic, amphoteric and/or nonionic surfactant;
d) water and/or a water-soluble solvent with hydroxy functionality
   and optionally auxiliary substances.

In a preferred embodiment, the cleaning composition can comprise a further amphiphilic substance.

Multi-component systems of the type of water, water-insoluble substance (oil), surfactant and optionally co-surfactant which form spontaneously and appear as multi-substance systems are known as microemulsions. Microemulsions are thermodynamically stable nanostructured fluids which comprise at least water or a water-like liquid (e.g. glycerol), oil and a surfactant. Microemulsions in some cases also contain co-surfactants and (if ionic surfactants are used) optionally also salts. The structure sizes of microemulsions are usually between 10 to 200 nm. In contrast to kinetically stable emulsions or nanoemulsions, the thermodynamically stable microemulsions do not tend to cream by particle coalescence. In microemulsions, larger structures formed in the short term disintegrate into smaller micelles again some time later. It follows from this that microemulsions are also formed by themselves due to their thermodynamic stability, without mixing. In contrast to emulsions, not only spherical micelles but also elongated micelles (worm-like micelles) and diverse network-like structures occur in microemulsions. In the most favourable case, a bicontinuous structure exists in a microemulsion. Here, the aqueous and oily phase penetrate through sponge-like interfaces of surfactants and optionally co-surfactants.

By the further addition according to the invention of amphiphilic substances, so-called NP-MCA (nanophase-forming mixed-chain structure amphiphiles), which do not follow the hydrophilic-hydrophobic structure or properties of surfactant or co-surfactant, an extension of the monophase colloidally disperse region of the microemulsion can be achieved and a modification of the properties can be established.

It has furthermore been found, surprisingly, that the addition of NP-MCAs has the effect of extending the thermodynamically stable monophase existence region of nanostructured systems. This was all the more surprising, since it was assumed hitherto in technical circles that the more different the lipophilic and the hydrophilic parts with respect to their solubility in the particular opposite phase, the more microemulsions can form. The person skilled in the art therefore in principle took oils and hydrophilic constituents which dissolve in one another as little as possible for the preparation of so-called microemulsions. Consequently, according to the prior art those substances which are not surface-active and nevertheless stay both in the oily phase and in the hydrophilic phase, as is the case with the amphiphiles according to the invention which do not form structures and are of mixed-chain structure (NP-MCA), were avoided.

In this respect, the present invention overcomes a prejudice which has been rooted in technical circles for a long time.

It was moreover surprising that the addition of NP-MCAs to an oil/water/surfactant mixture allows a significant widening of the monophase region of the nanophase fluids formed to develop compared with conventional microemulsions and, compared with conventional microemulsions, the lamellar phase (Lα) in a phase diagram called a fish diagram or “whale diagram” is suppressed a long way, so that the occurrence of highly viscous lamellar phases in which the oil and water domains are adversely present in layers is prevented or at least reduced (see FIG. 10).

It was also surprising that a lowering of the temperature window takes place due to the addition according to the invention of an NP-MCA, for example an ethyl acetoacetate, and therefore a larger usable temperature range can be achieved compared with conventional microemulsions (see FIG. 10).

In the context of the invention, these systems are called fluid nanophase systems (abbreviated to: nanophase fluids). Nanophase fluids contain water or a water-like substance, oil, at least one structure-forming amphiphile which accumulates at the oil-water interface and—in extension to the microemulsions—at least one further amphiphile which is not structure-forming and is without a surfactant structure (NP-MCA). The structure-forming amphiphile is a surfactant, co-surfactant or a surfactant-like oligomer or polymer. The NP-MCAs are important for the extension of the thermodynamically stable existence region of the fluid nanophases and therefore a further demarcation criterion from the microemulsions.

The addition of NP-MCAs renders possible a significant widening and optionally lowering of the temperature window of the monophase region. In addition, NP-MCAs prevent or reduce the occurrence of highly viscous lamellar phases, optionally reduce the surfactant concentration required and greatly extend the properties and possible uses of the fluids.

Nanophase-forming mixed-chain structure amphiphiles (NP-MCA) are mixed-chain structure amphiphiles which have hydrophilic and hydrophobic molecular regions which lie spatially close to one another but are mixed such that they have no surfactant-like structure. They therefore differ from surfactants and co-surfactants, which acquire their function by aligned separation of the two regions (head-tail structure). As a consequence of this, NP-MCA are not capable of formation of superstructures by themselves and do not accumulate preferentially at the oil-water interface. For formation of nanophase fluids, in addition to the oily or aqueous phase, a surfactant is therefore additionally also necessary. However, NP-MCA have a significant solubility in the aqueous phase or oily phase and become distributed therein until an equilibrium is formed. The solubility of the NP-MCA in water or oil is as a rule between 4 and 1,000 grams per litre, optionally also in the form of its salts.

An NP-MCA according to the invention is an amphiphilic substance which has no aligned hydrophilic-hydrophobic surfactant structure, is not structure-forming by itself, i.e. does not form micelles, the solubility of which in water or oil is between 4 g and 1,000 g per litre and which does not accumulate preferentially at the oil-water interface, with the proviso that the NP-MCA is not chosen from 2-ethyl-1,3-hexanediol, 2-methyl-2,4-pentanediol, 2-(n-butyl)-2-ethyl-1,3-propanediol and/or from 1,2-diols.

In microemulsions, in the phase diagram as a function of temperature and surfactant concentration (fish or whale diagram), a triangle can be stretched between the X point and the points of intersection of the boundary region of the monophase to the two-phase region and the tangent of the start of the Lα region laid parallel to the ordinate. Measurement methods for plotting the surfactant concentration/temperature phase diagram (fish or whale diagram) are known to the person skilled in the art from the prior art. NP-MCAs lead to a widening of the existence region of the monophase region, and to an increase in the area of this triangle, and can be defined by this means. NP-MCAs which can preferably be used are all amphiphiles which, on an addition of 4% to an oil-water-surfactant system, lead to an increase in the area of these triangles of at least 5%, without thereby changing the surfactant system, preferably of at least 10% and very particularly preferably of at least 20%. In a particular embodiment, the area of the triangle is increased in a range of from 5% to 2,000%, without the surfactant system thereby being changed, preferably from 10% to 1,000%, very particularly preferably from 15% to 500%.

Particularly preferred NP-MCA are characterized in that on an addition to an oil-water-surfactant system containing the constituents oil a), surfactant c) and water d) of 4 wt. %, based on the total weight of the system, they lead to an at least 5% increase in the area of the triangle contained in the phase diagram which is determined by the three corner points:

i) the X point,
ii) the upper point of intersection of the boundary region of the monophase to the two-phase region with the tangent to the start of the Lα region laid parallel to the temperature ordinate and
iii) the lower point of intersection of the boundary region of the monophase to the two-phase region with the tangent to the start of the Lα region laid parallel to the temperature ordinate.

The position of such triangles is illustrated in FIG. 10.

The method for plotting such phase diagrams is described, for example, in:
M. Kahlweit, R. Strey, D. Haase, H. Kunieda, T. Schmeling, B. Faulhaber, M. Borkovec, H. F. Eicke, G. Busse, F. Eggers, T. Funck, H. Richmann, L. Magid, O. Soderman, P. Stilbs, J. Winkler, A. Dittrich, and W. Jahn: “How to Study Microemulsions”, J. Colloid Interf. Sci., 118 (2), 436 (1987)
Microemulsions, T. Sottmann and R. Strey in Fundamentals of Interface and Colloid Science, Volume V, edited by J. Lyklema, Academic Press (2005).

To obtain a phase diagram (fish diagram or whale diagram), samples are prepared with a constant ratio of the non-surfactant components and a surfactant content which is increased stepwise starting from 0% up to a desired surfactant content (optionally up to 100%). The step width depends on the measurement accuracy requirements, a step width of 2% usually being adequate. These samples are left in a thermostatically controlled medium (preferably water, if necessary with additions which lower the freezing point) at temperatures of from −30° C. to 100° C. until phase equilibrium is established, and thereafter the phase state is evaluated visually via light scattering. The width of the temperature step results from the desired measurement accuracy, a step width of 1° C. usually being adequate for technical uses. The phase boundaries result from the transition from one phase state into the next, the error being determined by the step width of the temperature measurement. The measurement points obtained in this way are entered into a diagram and joined to one another, the temperature being plotted against the surfactant content. It is usually sufficient to discover the phase states existing in the measurement range in a sample and to determine the phase boundaries via a nest of intervals.

The value of the phase widening of the nanostructured fluid composition is determined by plotting a triangle in the phase diagram of FIG. 10 by a procedure in which a first straight line a) is formed starting from the X point to the curve characterizing the phase state above the middle temperature (line over 2), a second straight line b) is formed such that it touches the apex angle of Lα tangentially and intersects the first straight line a) at the site of its tangential point of contact with the curve characterizing the phase state above the middle temperature (line over 2), and a third straight line c) laid on the curve characterizing the phase state below the middle temperature (line under 2) such that it intersects the two straight lines a) and b). A numerical value A 1 results from totaling the lengths of the three straight lines in FIG. 10, which corresponds to a microemulsion according to the prior art. Analogous totaling of the lengths of the straight lines of a phase diagram according to the invention (nanophase fluid) gives a numerical value A2. The numerical value of the advantageous phase widening achieved by the present invention is determined by forming the numerical ratio of A2/A1, that is to say by dividing A2 by A1. For the composition according to the invention of the nanophase fluid, this numerical value is greater than 1.0; in particular greater than 1.1; especially greater than 1.15; very particularly greater than 1.2; preferably greater than 1.22. In this context, the influencing of the extent of the triangle can take place additionally or alternatively to the increasing of the area of the triangle.

Preferred NP-MCA are distinguished in that on an addition of 4 wt. %, based on the total weight of the cleaning composition, to an oil-water-surfactant system containing the constituents a), c) and d), they lead to an at least 5% increase in the temperature range ΔT of the monophase existence region of the cleaning composition, which is determined by the length, determined in the phase diagram as a function of temperature and surfactant concentration, of the tangent to the L_{α} region parallel to the temperature axis which is demarcated by the points of intersection of the tangent with the lower and upper separating line between the monophase and two-phase existence region of the cleaning composition (see FIG. 10). Particularly preferred NP-MCA lead to an increase in the temperature range ΔT of from 10% to 1,000%, very particularly preferably from 20% to 500%. In this context, the influencing of the temperature range ΔT can take place additionally or alternatively to the increasing of the area and/or extent of the triangle.

NP-MCA are molecules which comprise carbon, hydrogen and at least one of the following types of atom (hetero atoms): silicon, oxygen, nitrogen, sulphur, phosphorus, fluorine, chlorine, bromine, iodine. Polar carbon atoms are found alongside hetero atoms. Polar carbon atoms are not counted in an alkyl chain or non-polar chain.

NP-MCA in the context of the invention are above all to be found among the alcohols, ketones, esters, heterocyclic compounds having 5 to 7 atoms per ring, ethers, amides and amines, N-acylated amino acids and some aldehydes, which have no surfactant-like structure, that is to say no aligned head-tail structure.

These are, inter alia, alcohols (monoalcohols, dialcohols, trialcohols etc.) which have no surfactant-like structure. Hydrophilic and hydrophobic regions are mixed in the molecule such that:
i) no terminal non-polar chain on a primary or secondary carbon atom has 4 or more carbon atoms. Should the chain be longer, it must not make up more than 20% of the molecular weight;
ii) a non-polar chain inside the molecule or on a tertiary carbon atom is no longer than 7 carbon atoms (i.e. larger than e.g. 1,9-nonanediol) and does not make up more than 20% of the molecular weight. Larger chains are capable of being in the non-polar region, while the polar contents of the molecule are to be found in the hydrophilic region;
iii) in monocyclic alcohols, the shortest path through the ring is chosen as the chain length for determining the chain length according to point i and ii;
iv) in polycyclic alcohols, only the completely non-polar rings are taken into account for determination of the chain length corresponding to point i and ii, and the smallest number of carbon atoms is taken as the chain length here.

On the basis of comparable polarity, that said for alcohols applies analogously to amines and alcohol amines. The same applies to analogous fluorides, chlorides and molecules built up from such groups.

The present invention likewise provides a composition which comprises those amphiphiles from the group of alcohols, amines and alcohol amines which are not structure-forming and are of mixed-chain structure.

NP-MCA in the context of the invention can also be, in particular, ketones or acids and their weak salts and amides, and organyl sulphates and phosphates. On the basis of their somewhat higher polarity compared with alcohols, a chain length increased by 1 applies here to terminal chains and chains inside the molecule.

The present invention consequently likewise provides a composition which comprises those amphiphiles from the group of ketones or acids and their weak salts and amides, and organyl sulphates and phosphates, which are not structure-forming and are of mixed-chain structure.

NP-MCA in the context of the invention can also be alkyl, alkenyl, alkynyl, aryl sulphides, alkyl, alkenyl, alkynyl, aryl phosphides and alkyl-, alkenyl-, alkynyl-, arylsilicones/-siloxanes. On the basis of the lower polarity, a chain length reduced by 1 compared with alcohol applies here.

The present invention accordingly likewise provides a composition which comprises those amphiphiles which are not structure-forming and are of mixed-chain structure and have alkyl, alkenyl, alkynyl radicals or are from the group of aryl sulphides, aryl phosphides and arylsilicones/-siloxanes.

Furthermore, according to the invention those NP-MCAs which contain several of the abovementioned functionalities are also preferred in particular, it also being possible for various functional groups to occur in the molecule. The chain lengths stated for alcohols serve here as chain lengths for demarcation from conventional surfactant-like molecules, provided that the functionalities are not predominantly ketones, acids and their weak salts, amides or organyl sulphates or phosphates.

Preferred NP-MCA are chosen from diols of the formula I:

R₁R₂COH—(CH₂)ₙ—COHR₁R₂ [formula I]
wherein
n can be 0, 1, 2, 3 or 4,
R₁ and R₂ each independently of each other are hydrogen or an unbranched or branched C₁-C₃ alkyl, with the proviso that if n=0, R₁ cannot be hydrogen and the diol is not 2-methyl-2,4-pentanediol.

In particular, particularly preferred NP-MCA are chosen from the following diols: 1,3-propanediol, 1,3-butanediol, 1,4-butanediol, 1,5-pentanediol, 1,6-hexanediol, 2,3-butanediol, 2,4-pentanediol or 2,5-dimethyl-2,5-hexanediol.

The diols mentioned are suitable in particular for providing a cosmetics and/or dirt remover.

Preferred NP-MCA are chosen from acetoacetates of the formula II:

C(R₃)₃—CO—CH₂—CO—O—R₄ [formula II]
wherein
R₃ each independently of each other is hydrogen or a C₁ to C₂ alkyl and
R₄ is a branched or unbranched C₁ to C₄ alkyl;
   or from acetoacetates of the formula III:

CH₃—CO—CH₂—CO—O—R₅ [formula III]
wherein
R₅ is a C₁ to C₄ alkyl.

In particular, particularly preferred NP-MCA are chosen from the following acetoacetates: ethyl acetoacetate, iso-propyl acetoacetate, methyl acetoacetate, n-butyl acetoacetate, n-propyl acetoacetate or tert-butyl acetoacetate.

The acetoacetates mentioned are suitable in particular for providing a dirt remover, tile cleaner, cosmetics remover, hair decolorizer, graffiti remover, stripping formulation and/or nail varnish remover.

Further preferred NP-MCA are chosen from diones of the formula IV

CH₃—(CH₂)ₚ—CO—(CH₂)_{q}—CO—(CH₂)ᵣ—CH₃ [formula IV]
wherein
p, q, r independently of each other can be 0, 1 or 2, with the proviso that if the sum of p, q and r=2, the compound according to formula IV can also be cyclic (cyclohexanedione).

In particular, particularly preferred NP-MCA are chosen from the following diones: 2,3-butanedione (diacetyl), 2,4-pentanedione (acetylacetone), 3,4-hexanedione, 2,5-hexanedione, 2,3-pentanedione, 2,3-hexanedione, 1,4-cyclohexanedione or 1,3-cyclohexanedione.

The diones mentioned are suitable in particular for providing a cosmetics remover, dirt remover, nail varnish remover and/or graffiti remover.

NP-MCA which are likewise preferred are chosen from esters of the formula V

R₆—CO—O—R₇ [formula V]
wherein
R₆ is a ring bond to R₇, CH₃ or COCH₃ and
R₇ is (CH₂)₂—O— ring bond to R₆, (CH₂)₂—O—(CH₂)₃—CH₃, CH₂—CH₃ or CH₂—CH(CH₃)—O— ring bond to R₆.

In particular, particularly preferred NP-MCA are chosen from the following esters: (1-methoxy-2-propyl)acetate, (2-butoxyethyl)acetate, ethylene carbonate, ethyl pyruvate (2-oxopropionic acid ethyl ester) or propylene carbonate.

The esters mentioned are suitable in particular for providing a dirt remover, tile cleaner, cosmetics remover, hair decolorizer, graffiti remover, stripping formulation and/or nail varnish remover.

Further preferred NP-MCA are chosen from maleic or fumaric acid amides of the formula VI

R₈—HN—CO—C═C—CO—O—R₉ [formula VI]
wherein
R₈ is hydrogen, a branched or unbranched C₁-C₄ alkyl, or a branched or unbranched, linear or cyclic C₁-C₆ alkyl, wherein the C₁-C₆ alkyl is substituted by one or more groups chosen from OH, NH₂, COOH, CO, SO₃H, OP(OH)₂,
and R₉ is hydrogen or a branched or unbranched C₁-C₄ alkyl.

In particular, particularly preferred NP-MCA are chosen from the following maleic acid amides and methyl, ethyl, propyl and butyl esters thereof: N-methylmaleamide; N-ethylmaleamide; N-(n-propyl)-maleamide; N-(i-propyl)-maleamide; N-(n-butyl)-maleamide; N-(i-butylmaleamide); N-(tert-butylmaleamide), and the corresponding fumaric acid amides and methyl, ethyl, propyl and butyl esters thereof.

Further preferred NP-MCA are chosen from: 2,2-dimethoxypropane, pyruvic aldehyde 1,1-dimethyl acetal, diacetone alcohol (2-methyl-2-pentanol-4-one), 2-butanol, 2-acetyl-gamma-butyrolactone, 3-amino-1H-1,2,4-triazole, gamma-butyrolactone, nicotinamide, ascorbic acid, N-acetylamino acids, in particular N-acetylglycine, -alanine, -cysteine, -valine or -arginine, triethyl phosphate, n-butyl acetate, dimethylsulphoxide or 2,2,2-trifluoroethanol.

N-Acetylamino acids are suitable in particular for providing cosmetics removers.

The following NP-MCA are very particularly preferred according to the invention, these being chosen from the group consisting of ethyl acetoacetate; i-propyl acetoacetate; methyl acetoacetate; methyl isobutyrylacetate (methyl (4-methyl-3-oxopentanoate)); n-butyl acetoacetate; n-propyl acetoacetate; tert-butyl acetoacetate; allyl acetoacetate; maleic acid amide (maleamic acid, maleamide), the following maleamides and methyl, ethyl, propyl and butyl esters thereof: N-methylmaleamide; N-ethylmaleamide; N-(n-propyl)-maleamide; N-(i-propyl)-maleamide; N-(n-butyl)-maleamide; N-(i-butylmaleamide); N-(tert-butylmaleamide); and the corresponding fumaric acid amides and methyl, ethyl, propyl and butyl esters thereof; 2,2-dimethoxypropane; diacetone alcohol (4-hydroxy-4-methylpentan-2-one); 1,3-butanediol; 1,4-butanediol; 1,5-pentanediol; 1,6-hexanediol; 1,3-propanediol; 2,3-butanediol; 2,4-pentanediol; 2,5-dimethyl-2,5-hexanediol; (1-methoxy-2-propyl)acetate; (2-butoxyethyl)acetate; 1,3-cyclohexanedione; 1,4-cyclohexanedione; 2,3-hexanedione; 2,3-pentanedione; 2,5-hexanedione; 3,4-hexanedione; acetylacetone (2,4-pentanedione, ACAC); diacetyl (2,3-butanedione); ethylene carbonate; propylene carbonate; 2-acetyl-gamma-butyrolactone; N-acetylcysteine and the methyl, ethyl, propyl, butyl ester; N-acetylglutamic acid and the methyl, ethyl, propyl, butyl ester; N-acetylglycine and the methyl, ethyl, propyl, butyl ester; N-acetyltyrosine and the methyl, ethyl, propyl, butyl ester; N-acetylvaline and the methyl, ethyl, propyl, butyl ester; ethyl pyruvate (2-oxopropionic acid ethyl ester); pyruvic aldehyde 1,1-dimethyl acetal; 3-amino-1H-1,2,4-triazole; diethyl 3-oxoglutarate; diethylene glycol diethyl ether; diisopropyl ether; ethylene glycol diethyl ether; methyl carbamate; tert-butyl methyl ether; vinyl acetate; quinine (free base, as the hydrochloride); adipic acid diamide; succinic acid imide; N-methylcaprolactam; acetic acid diethylamide; urea; thioacetamide; 1,2-phenylenediamine; 1,3-phenylenediamine; 1,4-diaminobutane; 1,4-diazabicyclo[2.2.2]octane; 1,4-phenylenediamine; 1,6-diaminohexane; 2-(4-methoxyphenyl)-ethylamine; 2-aminobenzamide; 2-aminophenol; dipropylamine; triethylamine; tyramine; anthranilic acid; DL-2-aminobutyric acid; serine; threonine; tyrosine; adipic acid; methylenesuccinic acid; trans-propene-1,2,3-tricarboxylic acid; cyclohexanol; cyclohexanone; dimedone (5,5-dimethylcyclohexane-1,3-dione); N,N-dimethylcyclohexylamine; trans-1,2-cyclohexanediol; (4-hydroxyphenyl)-acetic acid; 1,3,5-trihydroxybenzene; 2-ethylpyridine; 2-methoxybenzoic acid; 2-methoxyphenol; 2-methylhydroquinone; 2-methylresorcinol; 2,4-dihydroxybenzoic acid; 2,6-dihydroxybenzoic acid; 3-aminophenol; 3,4-dihydroxybenzoic acid; 3,5-dihydroxybenzoic acid; 4-amino-3-nitrophenol; 4-aminophenol; 4-hydroxybenzaldehyde; 4-hydroxybenzoic acid; 5-methylresorcinol; acetylsalicylic acid; butylhydroxytoluene; N-phenyl-2,2′-iminodiethanol; N-phenylurea; methyl, ethyl, propyl 4-hydroxybenzoate; sulphanilic acid; vanillin; (2-ethoxyethyl)acetate; (2-ethoxyethyl)methacrylate; (2-hydroxypropyl)methacrylate; [2-(2-butoxyethoxy)-ethyl]acetate; 1,2-propylene glycol diacetate; diethyl malonate; dimethyl acetylsuccinate; dimethyl carbonate; dimethyl fumarate; dimethyl glutarate; dimethyl malonate; ethyl acetate; ethylene glycol diacetate; ethyl formate; ethyl lactate; glycerol triacetate; isopropenyl acetate; methyl formate; methyl lactate; methyl propionate; propyl formate; propyl propionate; tetraethyl orthocarbonate; triethyl citrate; 1-benzylpiperidin-4-one; 1-cyclohexyl-2-pyrrolidone; 1H-benzotriazole; 2-aminothiazole; 2-ethoxy-3,4-dihydro-2H-pyran; 2-ethylpiperidine; 2-mercapto-1-methylimidazole; 2-methyltetrahydrofuran; 2,2,6,6-tetramethyl-4-piperidinol; ascorbic acid; caffeine, theobromine, theophylline and the corresponding ethylxanthines; coumarine-3-carboxylic acid; ectoin; hydroxyproline; imidazole; indole; indole-3-acetic acid and its salts; melamine (2,4,6-triamino-1,3,5-triazine); methyl nicotinate; ethyl nicotinate, nicotinamide; nicotinic acid; pyridine-2-carboxylic acid; pyridine-2,3-dicarboxylic acid; pyridine-4-carboxylic acid; tropine (3-tropanol); tryptamine; nitroethane; nitromethane; 2-methyl-1-butanol; isobutanol (2-methyl-1-propanol); tert-amyl alcohol; 1,3-cyclopentanedione; 2,6-dihydroxyacetophenone; 3-methyl-3-penten-2-one; acetophenone; diethyl ketone; dihydroxyacetone; ethyl methyl ketone; isobutyl methyl ketone (methyl isobutyl ketone, MIBK); isopropyl methyl ketone; methyl propyl ketone; propiophenone; 2-butane oxime; sulphanilamide; 1,2,6-hexanetriol; 2-[4-(2-hydroxyethyl)-1-piperazinyl]-ethanesulphonic acid; 2-amino-2-methyl-1,3-propanediol (AEPD, ammediol), individually or as a mixture, including derivatives thereof.

The cleaning composition according to the invention preferably contains 1-80 wt. % of the NP-MCA, based on the total weight of the cleaning composition, particularly preferably 2-25 wt. %, very particularly preferably 10-24 wt. %.

For the purposes of the present invention, at least one water-insoluble substance with a solubility in water of less than 4 g per litre is understood as meaning oils. In this context, oil means all hydrophobic substances which do not mix homogeneously with water or water-like liquids and form a separate phase. Since some oils also dissolve in a large amount in water, a water-solubility of less than 4 grams per litre is additionally defined here. Preferably the water-insoluble substances are those with a water-solubility of less than 2 g per litre. These include e.g. alkanes (benzines) and cycloalkanes (preferably cyclohexane). Aromatics, such as toluene, xylenes or other alkylbenzenes and naphthalenes are also possible. Long-chain alkanoic acid esters, such as fatty oils and fatty acid alkyl esters or fatty alcohol ethers are preferred. According to the invention, benzyl acetate also belongs to the water-insoluble substances employed. Terpenes, e.g. monocyclic monoterpenes with a cyclohexane skeleton, however, can also be used. Terpenes from citrus fruits, such as lemon and/or orange terpenes and the limonene contained therein, are particularly preferred here. The cleaning compositions preferably contain 1-90 wt. % of the water-insoluble substances, particularly preferably 1.5-30 wt. %, based on the total weight of the cleaning composition.

Higher alcohols, for example, can be used as further amphiphilic substances. Above all co-surfactants with hydrophilic-lipophilic molecule contents, such as e.g. the n- and i-isomers of butanol, pentanol, hexanol, heptanol, octanol, nonanol, decanol, undecanol and dodecanol, are particularly preferred here.

Cycloalkanols are also preferred, such as cyclohexanol, or particularly preferably phenyl alcohols, such as phenylmethanol (benzyl alcohol), 2-phenylethanol and 3-phenyl-1-propanol.

Short-chain fatty acids, such as hexanoic, heptanoic, octanoic acid, and alkali metal or ammonium salts thereof can likewise be used. Salts thereof of ethanolamines are particularly preferred.

The composition according to the invention preferably contains from 2 to 45 wt. % of the further amphiphilic substances, based on the total weight of the cleaning composition, particularly preferably from 2 to 40 wt. %.

Particularly preferably, the further amphiphilic substance has a water-solubility of from 2 g to 128 g per litre and is chosen from the group comprising C₄-C₁₂-alcohols, cycloalkanols, phenyl alcohols, short-chain fatty acids or alkali metal or ammonium salts thereof.

The cleaning composition furthermore comprises anionic, cationic, amphoteric and/or nonionic surfactants. Some surfactants which are preferably suitable are mentioned in the following list.

Anionic surfactants which can be employed are e.g. alkali metal or ammonium salts of long-chain fatty acids, alkyl(benzene)sulphonates, paraffinsulphonates, bis(2-ethylhexyl) sulphosuccinate, alkyl sulphates, such as, above all, sodium dodecyl sulphate, and for specific uses where e.g. corrosion protection is important, in some cases also alkyl phosphates (e.g. Phospholan® PE 65, Akzo Nobel). Nonionic surfactants which can be used are polyalkylene oxide-modified fatty alcohols, such as e.g. Berol® types (Akzo-Nobel) and Hoesch T types (Julius Hoesch), and also corresponding octylphenols (Triton types) or nonylphenols (if the latter are not released into the environment in large amounts). A particular field of use is made possible by the heptamethyltrisiloxanes (e.g. Silwet® types, GE Silicones), as agents for greatly increasing the spreading properties of the liquids or significantly lowering the surface tension.

Cationic surfactants which can be used are e.g. coconut-bis-(2-hydroxyethyl)-methylammonium chloride or polyoxyethylene-modified trialkylmethylammonium chloride. In addition, the use of various amphoteric surfactants is also possible. If a wide pH range is to be covered, coconut-dimethylamine oxide (Aromox® MCD, Akzo-Nobel) has proved to be suitable.

The cleaning compositions preferably contain from 9 to 16 wt. % of the surfactants, based on the total weight of the cleaning composition.

Auxiliary substances which facilitate or improve use can optionally be added to the cleaning compositions according to the invention. This involves e.g. thickening to facilitate application of the cleaning composition to the substrate, lowering of flammability, improvement of smell, intensification of the action, reduction in the costs of the active compounds or corrosion protection. The sum of the auxiliary substances preferably makes up not more than 20 wt. % of the formulation, and the amount of an individual auxiliary substance is often not more than 10 wt. %.
a) Thickening: Most surfaces in a use are vertical walls from which low-viscosity liquids rapidly run off downwards from the site of damage by a graffito. Thickening of the liquids is therefore favourable, in order to delay or even prevent the running off. Commercially available thickening agents can be employed to thicken the low-viscosity liquids resulting from the experiments. Pyrogenic silica, such as e.g. Aerosil® 380, is particularly preferred.
b) Flameproofing: Since the graffiti remover can contain some highly volatile substances, a flameproofing agent may be appropriate. The composition can be formulated as self-extinguishing by addition of e.g. triethyl phosphate (TEP) or trioctyl phosphates, such as tris(2-ethylhexyl) phosphate (TEHP).
c) Improvement of smell: If the smell of the formulation is to be improved for the end user, this can be undertaken with a number of conventional aroma substances. Examples which may be mentioned are esters, such as methyl butanoate (pineapple), ethyl methanoate (raspberry), pentyl ethanoate (banana), pentyl pentanoate (apple), pentyl butanoate (apricot), octyl ethanoate (orange). The latter can also be combined well e.g. with orange terpenes or D-limonene, which can represent an example of a lipophilic active compound in the liquid according to the invention. Other aroma substances are also to be found in the field of terpenes, such as e.g. geraniol.

In a preferred embodiment the cleaning composition contains:
1-90% of water-insoluble substance a)
1-80% of NP-MCA b)
2-45% of surfactant c), including optionally further amphiphilic substance
1-90% of water
optionally auxiliary substances up to a max. of 10%
   the percentage data in each case relating to the total weight of the cleaning composition.

The composition according to the invention, which is a liquid comprising at least four but usually more components, surprisingly detaches lacquers and coatings significantly more effectively, more cleanly and in some cases more rapidly from various substrates than
a) conventional graffiti removers (cf. FIG. 1), stripping formulations or nail varnish removers on the conventional solvent basis or than solvents generally, even if these are known to have good solvating properties
b) the individual components per se (cf. FIG. 2)
c) mixtures of the solvent components without water and/or without surfactants
d) conventional graffiti removers on the known emulsion basis or the emulsions prepared from the solvent components
e) mixtures of water and surfactants.

Furthermore, these liquids dissolved the graffiti paints from the substrate at least equally as well as the most effective solvents of the solvents furthermore tested, that is to say namely e.g. tetrahydrofuran, 1-methyl-2-pyrrolidone, 1-methoxy-2-propanol, butyrolactone or methylene chloride.

The liquids according to the invention showed still further surprising effects with respect to their dissolving properties:
a) the primary cleaning effect did not consist a priori in a physical dissolving of the lacquer, since no lacquer binder is dissolved in a molecularly disperse form by the liquids, but in a
b) marked swelling of the lacquer, in some cases in a wave-like form, in some cases with microscopically small holes in the lacquer layer and a subsequent breaking up (fragmenting) of the lacquer layer into pieces of lacquer approx. 100 μm to 2 mm in size and
c) a migration under large areas of the lacquer layer with subsequent lifting of the layer.
d) All the treated, variously structured substrates showed practically no colour shadows when the lacquer was treated with damp cleaning tools or low pressure sprays (3-20 bar), since the liquids did not dissolve the lacquers in a molecularly disperse form and therefore released no finely divided pigment particles and, in combination with water, functioned as a washing liquor.

After an action time of only a few seconds to minutes it was possible to detach lacquers and coatings completely e.g. by means of a damp sponge, brush or paintbrush, an emulsion forming from the previously transparent liquid in combination with water, which indeed had a significantly low detaching power, but in return a pronounced washing action, and removed the coloured particles and binder residues from the substrate. Due to this washing action, no fine coloured gradient (“shadow”), which conventional solvent (mixtures) usually leave on the substrate from the site of use to untreated areas and which consists of diluted binders and pigments, was formed,

The cleaning effect of the liquids according to the invention thus comprises two stages:
a) in undiluted form: swelling or superficial dissolving of the binder systems of the graffiti paint, usually in combination with fragmenting of the lacquer—in the case of swelling also migration under the paint layer to the substrate
b) together with small amounts of water such as occur e.g. in a damp sponge, brush or paintbrush: formation of a washing liquor and lifting of the swollen and/or dissolved lacquer particles from the substrate.

In contrast to lacquer removers to date on a conventional solvent basis, no colour gradient, i.e. smearing of the dissolved graffiti paint—neither towards the edges of the coatings nor into the substrate—therefore results on the areas treated according to the invention. The photography in Fig. of a treated coarse rendering substrate shows this action compared with a conventional graffiti remover based on 1-methoxy-2-propanol. The photography in FIG. 2 shows the same action on removal of nail varnish.

In contrast to graffiti removers to date on a conventional solvent basis, the systems according to the invention show a very broad action spectrum on the detachment of paints or coatings, without the limitations to certain binder types such as is often the case when solvents are used. The graffiti remover system according to the invention shows a selectivity with respect to emulsion paints, which produce coloured coverings by coalescence of very fine highly polymeric particles and are often employed as façade paints, i.e. high-quality façade paints are not detached within the action time. In the case of stripping formulations, this selectivity is not necessary, since here also substrate coatings must also be lifted at the same time.

In contrast to lacquer removers on the conventional solvent basis, the systems according to the invention show a very rapid swelling and detachment of the paints, depending on age, substrate and paint conventionally within 10 seconds to about 30 minutes (preferably 20 minutes) for graffiti removers, 20 minutes to 3 hours for stripping formulations and 3 to 30 seconds (preferably 3 to 20 seconds) for nail varnish removers.

The invention thus also relates to the use of cleaning compositions according to the invention as graffiti removers, stripping formulations or nail varnish removers.

The invention also provides a method for the removal of undesirable paints and lacquers from surfaces. The process according to the invention for the removal of undesirable paints and lacquers from surfaces is distinguished in that the cleaning composition according to the invention is applied to the undesirable paint or the lacquer, acts, and the paint or the lacquer is then removed with water, the action time being from about 10 seconds to about 30 minutes for graffiti removers, from about 20 minutes to about 3 hours for stripping formulations and from about 3 to about 30 seconds for nail varnish removers.

The invention furthermore relates to the use of cleaning compositions according to the invention as dirt removers, tile cleaners, cosmetics removers or hair decolorizers without oxidative bleaching. In this context, dirt is understood as meaning the presence of at least one component chosen from carbon black, fat, oil, silicone, fine dust, resin and/or mixtures containing one or more of these constituents. Cosmetics are understood as meaning compositions for body and beauty care, in particular those compositions which are applied to the skin and/or cutaneous appendages, such as, for example, hair or nails. Hair is understood as meaning both artificial hair and real natural hair. Hair colours are understood as meaning compositions which colour the hair without bleaching it oxidatively.

The invention also provides a method for the removal of dirt (for example carbon blacks, fats, oils, silicones, fine dusts, resins and mixtures containing one or more of these constituents) from surfaces, such as, for example, ceramic, tile or plastics surfaces, of cosmetics or for decolorizing hair, characterized in that a cleaning composition according to the invention is applied to the dirt, the cosmetics to be removed or the hair to be decolorized, acts, and the dirt, the composition or the colour is then removed with water, the action time being from about 10 seconds to about 3 hours for dirt removers, from about 10 seconds to about 30 minutes for cosmetics removers and from about 2 minutes to about 24 hours for hair decolorizers.

The invention furthermore relates to a process for the preparation of the cleaning composition according to the invention. The process according to the invention for the preparation of a cleaning composition is distinguished in that water or a solvent with hydroxy functionality is initially introduced into a vessel and an anionic, cationic, amphoteric and/or nonionic surfactant is dissolved therein at 10 to 90° C., while stirring, water-insoluble substance(s) are added in parallel with or after the addition of surfactant and the emulsion formed is then converted into a visually transparent extended microemulsion or a nanophase system by the addition of a further amphiphilic substance and NP-MCA, and auxiliary substances are optionally added at the end of the mixing operation.

The cleaning composition is prepared by initially introducing first water or the solvent with hydroxy functionality into a suitable vessel and then dissolving the surfactant, while stirring. It is to be noted here that some surfactants can already contain water in the delivery form, so that the amount of water calculated in advance in the recipe may have to be corrected. When dissolving the surfactant, it must be ensured that the introduction of air into the solution is kept as low as possible in order to avoid excessive foaming. For realization on a large industrial scale, there are already many variations of stirring apparatuses and stirrers for largely avoiding foaming. If propeller stirrers and ideal ratios of stirrer diameter and vessel diameter are used, the stirring speed should conventionally not exceed 200 revolutions per minute. It must furthermore be remembered that some (concentrated) surfactants can form gels on addition of water, which can make stirring and further distribution difficult. In such cases, where appropriate the water-insoluble substances (oily phase) must be added first or in parallel with the addition of surfactant. Foaming can also be prevented by subsequent addition of the oily phase, since this often has a certain defoamer action. After addition of the oily phase, a milky-cloudy emulsion has formed, which clarifies by the addition of the further amphiphilic substance with a surfactant structure (e.g. alkanol), but at the latest after addition of the amphiphile according to claim 1b (e.g. the acetoacetate compound) and finally converts into a visually transparent extended microemulsion or a nanophase system. At the end, additives, such as e.g. flame retardant agents (e.g. triethyl phosphate), thickening agents (e.g. Aerosils) and/or other auxiliary substances can also be added.

The invention also provides a process for the preparation of a cleaning composition according to the invention, characterized in that water or a solvent with hydroxy functionality is initially introduced into a vessel and an anionic, cationic, amphoteric and/or nonionic surfactant is dissolved therein at 10 to 90° C., while stirring, water-insoluble substance(s) are added in parallel with or after the addition of surfactant and the emulsion formed is then converted into a visually transparent microemulsion or a nanophase system by the addition of a further amphiphilic substance and NP-MCA, and auxiliary substances are optionally added at the end of the mixing operation.

The figures show

FIG. 1: Comparison of the graffiti remover according to the invention according to Example 2 with a conventional graffiti remover based on 1-methoxy-2-propanol (Baufan RICO Graffiti-Killer)

Photograph of a wall with coarse rendering damaged by graffiti (age of the graffito at least 1 year). Action of the graffiti removers at the same time and for the same length of time, wiping off of the removers with a sponge after 5 minutes and subsequent rinsing of the wall with a low pressure water jet (<3 bar):
a) The formulation according to the invention shows no colour shadow, no impairment of the original façade paint (white) and no colour gradient at the edges, but a sharp demarcation from the untreated area.
b) The conventional graffiti remover based on 1-methoxy-2-propanol, on the other hand, shows a poorer removal of the paint with blurred edges from the site of action to the untreated area, which is caused by smearing of the graffiti paint. Furthermore, the paint diluted by the composition has been partly absorbed by the pores of the plaster, which produces colour shadows which can be removed subsequently only with difficulty.

FIG. 2: Activity of the conventional nail varnish remover Essence® with ethyl acetate as the lacquer solvent, of the nail varnish remover according to the invention of Example 5 and of individual components.
a) good removal of varnish, smearing towards the edge
b) good removal of varnish by the nanophase fluid, sharp edges with swollen regions
c) virtually no detachment, no swelling
d) hardly any removal of varnish
e) removal of varnish with severe smearing towards the edge

The individual components c) and d) of the nanophase fluid b) show in themselves no activity on the detachment of the varnish; component e) is a poor solvent.

FIG. 3: Activity of cleaning compositions according to the invention as dirt removers with a) condition before cleaning and b) condition after cleaning. A commercially available cleaning composition was used under 1 and a cleaning composition according to the invention with nanophase structuring was used under 2.

FIG. 4: Activity of cleaning compositions according to the invention as cosmetics removers with a) condition before cleaning and b) condition after cleaning. A commercially available cosmetics remover was used under 1 and a cleaning composition according to the invention with nanophase structuring was used under 2. The figures right at the bottom in b) indicate the cleaning cycles, i.e. how many times the area was wiped over with gentle pressure and only in one direction with a cotton-wool pad impregnated with the corresponding cleaning composition.

FIG. 5: Activity of cleaning compositions according to the invention as hair decolorizers with a) condition after cleaning and b) condition before cleaning.

FIG. 6: Scattering of a laser beam for detection of the nanostructuring in liquid systems with a) ethyl acetoacetate, b) acetone, c) cleaning composition according to the invention graffitiCRACK, d) cleaning composition according to the invention LisoCLEAR, e) cleaning composition according to the invention V1113.

FIG. 7: Activity of cleaning compositions according to the invention in the removal of lacquer in comparison with cleaning compositions which do not form nanophases, the results after cleaning being shown for a) ethyl acetoacetate, b) V141 (no nanophase), c) cleaning composition according to the invention NP1, d) cleaning composition according to the invention NP2, e) cleaning composition according to the invention NP3, V142 (no nanophase).

FIG. 8: Activity of cleaning compositions according to the invention in cleaning of tiles in comparison with cleaning compositions which do not form nanophases, in each case showing in a) the condition before cleaning and in b) the condition after cleaning. The results for a microemulsion are shown under 1), the results for an emulsion system are shown under 2) and the results of a nanophase system according to the invention are shown under 3).

FIG. 9: In FIG. 9, the nanostructuring of the fluid NP2 can be seen by means of a freeze fracture electron microscopy photograph. The smaller spherical structures (arrow) are micelles of the aqueous phase approx. 20-50 nm in size which are distributed within a little-structured oily phase.

FIG. 10: Phase diagram (fish diagram or whale diagram) which represents the course of the monophase and two-phase and lamellar existence regions of a cleaning composition as a function of the surfactant concentration and the temperature. A composition as a microemulsion is shown in a), and the same composition additionally containing 4% of NP-MCA, as a nanophase fluid, is shown in b). The temperature range, ΔT, of the monophase existence region of the cleaning composition is shown, ΔT being determined by the length, determined in the fish diagram, of the tangent to the L_{α} area parallel to the temperature axis which is demarcated by the points of intersection of the tangent with the lower and upper separating line between the monophase and two-phase existence region of the cleaning composition. As can be seen from FIG. 10, the presence of NP-MCA leads to an increase in the temperature range ΔT.

The following embodiment examples are intended to explain the invention in more detail without limiting it thereto.

### EMBODIMENT EXAMPLES

### Example 1

### Graffiti Remover

| Substance | Percent by weight [%] |
|---|---|
| Water, demineralized | 20.00 |
| Triethyl phosphate | 4.50 |
| Ethyl acetoacetate | 17.20 |
| Orange terpenes | 30.00 |
| Sodium lauryl sulphate | 12.30 |
| 1-Hexanol | 16.00 |
| | 100.00 |

The graffiti remover from Example 1 was prepared by first initially introducing water into a suitable vessel and dissolving the surfactant (sodium lauryl sulphate) therein, while stirring. During dissolving of the surfactant, the introduction of air into the solution should be kept as low as possible. Foaming can be prevented by subsequent addition of the orange terpenes, since these have a certain defoamer action. After this step, a milky-cloudy emulsion is formed, which becomes clear by addition of 1-hexanol and ethyl acetoacetate and finally converts into a completely transparent nanophase system. At the end, the triethyl phosphate is added.

### Example 2

### Graffiti Remover

| Substance | Percent by weight [%] |
|---|---|
| Water, demineralized | 24.50 |
| Triethyl phosphate | 5.00 |
| Ethyl acetoacetate | 14.50 |
| Orange terpenes | 27.50 |
| Sodium olefinsuiphonate (Hansanyl OS) | 12.50 |
| 1-Hexanol | 16.00 |
| | 100.00 |

The graffiti remover from Example 2 was prepared by a process analogous to Example 1.

### Example 3

### Graffiti Remover

| Substance | Percent by weight [%] |
|---|---|
| Water, demineralized | 32.00 |
| Triethyl phosphate | 4.50 |
| Ethyl acetoacetate | 11.50 |
| n-Butyl acetate | 8.00 |
| Orange terpenes | 12.00 |
| Benzyl acetate | 8.00 |
| Sodium dodecyl sulphate | 11.00 |
| 1-Hexanol | 13.00 |
| | 100.00 |

The graffiti remover from Example 3 was prepared by a process analogous to Example 1.

### Example 4

### Stripping Formulation

| Substance | Percent by weight [%] |
|---|---|
| Water, demineralized | 30.77 |
| Triethyl phosphate | 4.32 |
| Ethyl acetoacetate | 11.06 |
| Dibase Ester (DuPont) | 7.69 |
| Benzyl acetate | 7.69 |
| Orange terpenes | 15.39 |
| Hoesch NAS (as 100% strength substance) | 10.58 |
| 1-Hexanol | 12.50 |
| | 100.00 |

The stripping formulation from Example 4 was prepared by a process analogous to Example 1.

### Example 5

### Nail Varnish Remover

| Substance | Percent by weight [%] |
|---|---|
| Water, demineralized | 43.49 |
| Ethyl acetoacetate | 22.26 |
| 2-Phenylethanol | 14.84 |
| Essential oil, oranges (fragrance) | 2.45 |
| Sodium lauryl sulphate | 16.96 |
| | 100.00 |

The nail varnish remover from Example 5 was prepared by trickling the surfactant (sodium lauryl sulphate) into a mixture of water, ethyl acetoacetate and 2-phenylethanol while stirring gently at stirring speeds of 100 revolutions per minute. After formation of the nanophase system, the fragrance was added.

The following overview shows the action of the graffiti removers according to the invention in comparison with conventional graffiti removers or solvents.

TABLE 1
Action of the graffiti removers of the invention prepared in Examples 1 and 3 in the
course of an action time of 5 minutes

| | | | | Graffiti remover | |
|---|---|---|---|---|---|
| | | | | based on 1- | |
| | | Example | Example | methoxy-2- | |
| Colour | Substrate | 1 | 3 | propanol | 2-Butanol |
| black | fine stone, | complete | complete | colour shadow | scarcely removal |
| | rough, microporous | | | | |
| black | | complete | complete | no removal in | no removal in |
| | | | | pores | pores |
| black | | complete | complete | complete | scarcely removal |
| black | | complete | complete | colour gradient | approx. 80% |
| | | | | | removal |
| black | | complete | complete | complete | scarcely removal, |
| | | | | | smearing |
| red | | complete | complete | colour shadow | scarcely removal |
| dark red | rendering painted | complete | complete | colour shadow | no result available |
| | white | | | | |
| yellow | | complete | complete | almost complete | |
| dark green | | complete | complete | | |
| | | complete | complete | | |
| turquoise green | | complete | complete | | |
| dark blue | | complete | complete | | |
| blue | | complete | complete | | |
| blue | | complete | complete | | |
| gold, metallic | | complete | complete | | almost complete |
| silver, metallic | | complete | complete | | almost complete |

TABLE 2
Action of the nail varnish remover according to the invention according to Example 5:
after a single application of the remover with cotton-wool pads and subsequent rinsing of
the nails with water

| | | | | “essence pocket |
|---|---|---|---|---|
| Nail varnish | | Example | | beauty” |
| colour | Brand | 5 | Acetone | Ethyl acetate |
| dark violet | BIG BLUE | detachment after 3 seconds | complete, but with | dissolves after 3 |
| | | | smears of colour on | seconds, smears of |
| | | | skin | colour on skin |
| brown-gold | BIG BLUE | detachment after 3 seconds, | no complete removal | dissolves after 3 |
| | | complete after approx. 10 | of the varnish from | seconds, smears of |
| | | seconds | the skin and nails | colour on skin |
| pink | essence | complete after approx. 10 | complete | complete |
| | cosnova | seconds | | |
| orange | 60 seconds, | after 15 seconds initial | complete | complete |
| | Rimmel | dissolving time, then rapid and | | |
| | | complete | | |
| dark red | Jade- | soluble immediately, can be | smearing | complete, but with |
| | Maybelline | removed without residues | | smearing |
| dark red | NIVEA | complete | smearing | complete |
| white | Luminelle, | after 10 seconds initial | almost complete | complete |
| | Yves Rocher | dissolving time, then within 10 | | |
| | | seconds fast and complete | | |

### Example 6

### Dirt Remover

Background: Removal of dirt from various substrates is still a problem which has been only partly solved. In many cases commercially available cleaning compositions are not able to penetrate into micrometre-sized pores and to dissolve out dirt particles or otherwise detach firmly adhering dirt. Nanostructured liquids, on the other hand, are capable of doing so due to the low surface tension, the property of creeping and the tendency to incorporate nano- and microparticles.

An air outlet box which had accumulated the secretions of birch trees (birch resin) on its white coat of lacquer over a period of approx. 10 years in the open air was chosen as an example. Commercially available cleaning compositions did not result in successful cleaning. Both compositions were allowed to act for 2 minutes and were then rinsed off with damp sponges.

FIG. 3 a) beforehand, b) after cleaning.

Pictures left-hand side (1): commercially available surfactant cleaning compositions do not produce a cleaning effect.

Pictures right-hand side (2): after cleaning with the nanostructured formulation, the birch resin has been detached without residues.

Composition of the fluid for cleaning (lisoCLEAR 55 DAA—the formulation was developed for cleaning tiles, ceramic, façades and lacquer):

| | | |
|---|---|---|
| Aqueous phase: | Water | 55.28% |
| Oily phase: | Orange terpene | 11.35% |
| Surfactant: | Sodium dodecyl sulphate | 8.80% |
| | C9-C11 alcohol ethoxylate (4) | 8.82% |
| Co-surfactant: | | |
| NP-MCA: | Diacetone alcohol (DAA) | 3.47% |
| | Ethyl acetoacetate | 12.28% |
| | | 100.00% |

### Example 7

### Tile Cleaner

Removal of dirt from fine-pored, ceramic or mineral substrates, such as fine stone tiles, rendering, concrete etc., is still a problem which has been only partly solved. In many cases commercially available cleaning compositions are not able to penetrate into micrometre-sized pores and to dissolve out dirt particles or otherwise detach firmly adhering dirt. Nanophase fluids, on the other hand, are capable of doing so due to the low surface tension, the property of creeping and the tendency to incorporate nano- and microparticles.

Ceramic tiles in a 100 year-old Art Nouveau house which had absorbed dirt over decades were chosen as an example. This dirt could not be removed by regular weekly cleaning with the recommended dosages of commercially available cleaning compositions.

All the compositions investigated were allowed to act for exactly 120 seconds and were then rinsed off with a damp sponge. Thereafter, after-cleaning was carried out two more times with water without pressure. In spite of having approximately the same constituents, a different action results, depending on whether the composition is a microemulsion, a conventional emulsion or a nanophase fluid.

The results are shown in FIG. 8, where
a) shows the condition before cleaning, with clearly visible dirt in the grooves of the tiles, and
b) shows the condition of the same tile after the cleaning.

The following compositions of the fluids were used here for cleaning the tiles:

composition 1. is a microemulsion,
composition 2. is an emulsion system and
composition 3. is the nanophase system lisoCLEAR 55 nPMA (the formulation was developed for cleaning tiles, ceramic, façades and lacquer).

The constituents of the compositions used are given in the following.

### Composition 1. Microemulsion as a Comparison System for Cleaning Tiles

| | | |
|---|---|---|
| Aqueous phase: | Water | 55.58% |
| Oily phase: | Orange terpene | 27.25% |
| Surfactant: | Sodium dodecyl sulphate | 2.50% |
| | C9-C11 alcohol ethoxylate (4) | 14.67% |
| Co-surfactant: | — | |
| NP-MCA: | — | |
| | | 100.00% |

### Composition 2. Emulsion System as a Comparison System for Cleaning Tiles

| | | |
|---|---|---|
| Aqueous phase: | Water | 55.52% |
| | 1-Methyl-2-pyrrolidone | 1.85% |
| Oily phase: | Orange terpene | 2.96% |
| Surfactant: | Sodium dodecyl sulphate | 23.09% |
| | C9-C11 alcohol ethoxylate (4) | 7.03% |
| Co-surfactant: | — | |
| NP-MCA: | Ethyl acetoacetate | 9.55% |
| | | 100.00% |

Composition 3. Nanophase Fluid lisoCLEAR 55 nPMA

| | | |
|---|---|---|
| Aqueous phase: | Water | 55.28% |
| Oily phase: | Orange terpene | 11.35% |
| Surfactant: | Sodium dodecyl sulphate | 8.80% |
| | C9-C11 alcohol ethoxylate (4) | 8.82% |
| Co-surfactant; | — | |
| NP-MCA: | Ammonium n-propylmaleamide | 3.47% |
| | (=maleic acid mono-n-propylamide | |
| | ammonium salt) | |
| | Ethyl acetoacetate | 12.28% |
| | | 100.00% |

### Example 8

### Cosmetics Remover

Background: Waterproof kiss- and tear-proof cosmetics are to be found on the market ever more frequently, and furthermore the quality has become ever better in recent times. Such cosmetics also can no longer be removed easily with warm water.

However, many make-up compositions disadvantageously comprise two phases which must be shaken shortly beforehand in order to form an emulsion. After often less than one or two minutes the phases separate again (the oily phase forms a cream).

The following nanophase fluid comprises very gentle, skin-friendly constituents which in themselves individually have an only weak cleaning action, if any. In the form of a nanostructured system, however, the action exceed that of the commercially available products.

FIG. 4 on the left (I): a) beforehand, b) after cleaning with Balea 2-Phase Eye Makeup Remover Waterproof.

Picture on the right (II): a) beforehand, b) after cleaning with the nanostructured formulation.

The figures right at the bottom in picture b) indicate the cleaning cycles, i.e. how many times the area was wiped over with gentle pressure and only in one direction (towards the fingers) with a cotton-wool pad impregnated with the cleaning composition.

Composition of the fluid for removal of cosmetics (“superformulation”):

| | | |
|---|---|---|
| Aqueous phase: | Water | 46.65% |
| | Sodium chloride | 0.43% |
| | Glycerol | 0.47% |
| | Glycine | 0.35% |
| Oily phase: | Dicaprylyl ether | 17.76% |
| | Oleyl oleate | 1.99% |
| Surfactant: | Polyethylene glycol-7 glyceryl cocoate | 11.08% |
| | Polyoxyethylene (4) sorbitan monostearate | 7.82% |
| Co-surfactant: | — | 0.00% |
| NP-MCA: | Ethyl acetoacetate | 12.25% |
| | Nicotinamide | 0.31% |
| | Ascorbic acid | 0.39% |
| | N-Acetylglycine | 0.50% |
| | | 100.00% |

### Example 9

### Decolorizing of Hair

A tuft of hair coloured with black hair colour was kept in fluid 42 overnight (16.5 hours). The decolorized hairs (a) were compared with the untreated tuft of hair (b): The results are shown in FIG. 5. It is found that decolorizing of the treated hairs has taken place.

### Composition of Fluid V42:

| | | |
|---|---|---|
| Aqueous phase: | Water | 28.48% |
| | Ethanol | 6.15% |
| Oily phase: | Orange terpene | 20.49% |
| Surfactant: | Cocoamidopropylbetaine | 6.05% |
| | (Tego Betain CK D) | |
| | C9-C11 alcohol ethoxylate (4) | 15.37% |
| | (Berol 260) | |
| Co-surfactant: | 1-Hexanol | 2.15% |
| NP-MCA: | Ethyl acetoacetate | 21.31% |
| | | 100.00% |

### Example 10

### Detection of the Fluid Nanophases

Experiments on the scattering of a laser beam for detection of nanostructuring in nanophase systems

The results are shown in FIG. 6:
a) Ethyl acetoacetate: green laser beam not visible in the liquid, i.e. no scattering and therefore no nanostructuring.
b) Acetone: green laser beam not visible in the liquid, i.e. no scattering and therefore no nanostructuring.
c) graffitiCRACK: green laser beam is visible by scattering, i.e. the liquid is nanostructured. A red laser beam moreover is scarcely scattered, since the wavelength of red light is to long for an interaction here.
d) lisoCLEAR: green laser beam is visible by scattering, i.e. the liquid is nanostructured.
e) Nail varnish remover (recipe V113): green laser beam is visible by scattering, i.e. the liquid is nanostructured.

Recipe for c): Nanophase fluid NP 2 (graffitiCRACK liquid):

| | | |
|---|---|---|
| Aqueous phase: | Water | 21.04% |
| Oily phase: | Orange terpene | 10.63% |
| | Benzyl acetate | 10.52% |
| Surfactant: | Sodium dodecyl sulphate (SDS) | 13.18% |
| | C9-C11 alcohol ethoxylate (4) | 2.13% |
| Co-surfactant: | n-Hexanol | 10.52% |
| NP-MCA: | Triethyl phosphate | 5.15% |
| | Ethyl acetoacetate | 18.38% |
| | n-Butyl acetate | 8.45% |
| | | 100.00% |

Recipe for d): lisoCLEAR 55 DAA (high-performance cleaner for tiles, façades, stone etc.

| | | |
|---|---|---|
| Aqueous phase: | Water | 55.28% |
| Oily phase: | Orange terpene | 11.35% |
| Surfactant: | Sodium dodecyl sulphate | 8.80% |
| | C9-C11 alcohol ethoxylate (4) | 8.82% |
| Co-surfactant: | | |
| NP-MCA: | Diacetone alcohol | 3.47% |
| | Ethyl acetoacetate | 12.28% |
| | | 100.00% |

Recipe for e): Nail varnish remover V113g

| | | |
|---|---|---|
| Aqueous phase: | Water | 41.30% |
| Oily phase: | Dicaprylyl ether | 3.45% |
| | Ethyl cinnamate | 0.42% |
| Surfactant: | Sodium dodecyl sulphate (SDS) | 4.20% |
| | C13 alcohol ethoxylate (3) | |
| | (Lutensol TO 3) | 9.81% |
| Co-surfactant: | 2-Phenylethanol | 3.46% |
| NP-MCA: | Diacetone alcohol | 11.74% |
| | Ethyl acetoacetate | 25.62% |
| | | 100.00% |

### Example 11

### Comparison Tests of Nanophase Fluids Vs. Microemulsions in a Lacquer Removal Experiment

For a comparison test in the detachment/dissolving properties of lacquers on a porous substrate, 6 liquids were tested:

a) Ethyl acetoacetate with an action as a solvent—not nanostructured: smearing of the lacquers.
b) Solvent mixture (V141)—not nanostructured: smearing of the lacquers
Glycerol: 8.34%, ethanol: 8.30%, diacetone alcohol: 35.02%, ethyl acetoacetate: 21.00%, n-butyl acetate: 6.64%, hexanol: 8.26%, benzyl acetate: 8.30%, orange terpene: 4.15%.
c) Nanophase fluid NP1 (V138d)—almost analogous to b), but nanostructured: no smearing, but fragmenting of the lacquers
Aqueous phase: Glycerol: 13.18%, Ethanol: 13.76%;
Oily phase: Dicaprylyl ether: 17.42%
Surfactant: Sodium dodecyl sulphate (SDS) 13.18%
   C9-C11 alcohol ethoxylate (4) 2.13%;
Co-surfactant:
NP-MCA: Diacetone alcohol: 8.741%, Acetylacetone: 8.71%.
   d) Nanophase fluid NP 2 (graffitiCRACK liquid): fragmenting of the lacquer, very good detachment of the lacquer
Aqueous phase: Water: 21.04%;
Oily phase: Orange terpene: 10.63%, Benzyl acetate: 10.52%;
Surfactant: Sodium dodecyl sulphate (SDS): 13.18%,
   C9-C11 alcohol ethoxylate (4): 2.13%;
Co-surfactant: n-Hexanol: 10.52%;
NP-MCA: Triethyl phosphate: 5.15%, Ethyl acetoacetate: 18.38%, n-Butyl acetate: 8.45%.
   e) Nanophase fluid NP1 (V143)—almost analogous to f), but nanophase fluid: more powerful action than f)
Aqueous phase: Water: 27.11%;
Oily phase: Orange terpene: 24.46%;
Surfactant: Sodium dodecyl sulphate (SDS): 8.92%,
   C9-C11 alcohol ethoxylate (4): 21.77%;
Co-surfactant:
NP-MCA: Ethyl acetoacetate: 17.75%.
   f) Microemulsion ME 1 (V142): nanostructured, but as a microemulsion slower in detaching the lacquer
Aqueous phase: Water 33.87%;
Oily phase: Orange terpene 34.21%;
Surfactant: SDS 11.34%;
Co-surfactant: Hexanol 20.58%

The liquids were applied to the porous reverse of a ceramic plate on to which stripes of the following lacquers were applied:

red: Product Auto K (20330, VW/Audi, mars red, L31 B)
yellow: Product Auto K (22218, FORD, signal yellow, 77 KLP/97
green: Product Auto K (21395, OPEL, mint green, 361)
silver: Product Monex, Lack Spray (Rallye wheel rim silver, 7093)
blue: Product Dupli Color, (sky blue, satin mat, DCP 5200/RAL 5015)

After application of the liquids, they were rubbed in with a paintbrush for 2 minutes and then rinsed off with running water.

The results are summarized in FIG. 7. The liquids which were not nanostructured indeed dissolved the lacquers, but also smeared significantly. On the other hand, none of the nanostructured systems shows smearing.

In the nanostructured liquids there is in turn a difference to be seen as to whether it is a microemulsion or a phase-widened system (nanophase fluid). The microemulsion showed the lowest detachment capability with respect to time. graffitiCRACK had the most significant action.

### Example 12

### Further Selected Formulation Examples for Cleaning Compositions According to the Invention

### Formulation 1:

| | Content [%] |
|---|---|
| Water | 19.80 |
| Triethyl phosphate | 4.85 |
| Ethyl acetoacetate | 17.30 |
| n-Butyl acetate | 7.95 |
| 1-Hexanol | 9.90 |
| Benzyl acetate | 9.90 |
| Orange terpene | 10.00 |
| Sodium dodecyl sulphate | 12.40 |
| C9-C11 | 2.00 |
| alcohol ethoxylate (4) | |
| Aerosil | 5.90 |

### Formulation 2:

| | Content [%] |
|---|---|
| Water | 44.00 |
| N-Methyl-2-pyrrolidone | 4.50 |
| Ethyl acetoacetate | 16.00 |
| Orange terpene | 15.00 |
| Sodium dodecyl sulphate | 11.50 |
| C9-C11 | 9.00 |
| alcohol ethoxylate (4) | |

### Formulation 3:

| | Content [%] |
|---|---|
| Water | 55.28 |
| N-Methyl-2-pyrrolidone | 3.47 |
| Ethyl acetoacetate | 12.28 |
| Orange terpene | 11.35 |
| Sodium dodecyl sulphate | 8.80 |
| C9-C11 | 8.82 |
| alcohol ethoxylate (4) | |

### Formulation 4:

| | Content [%] |
|---|---|
| Water | 28.48 |
| Ethanol | 6.15 |
| Ethyl acetoacetate | 21.31 |
| Orange terpene | 20.49 |
| Hexanol | 2.15 |
| Tego Betain CK D | 6.05 |
| C9-C11 | 15.37 |
| alcohol ethoxylate (4) | |

### Formulation 5:

| | Content [%] |
|---|---|
| Water | 41.32 |
| Diacetone alcohol | 11.73 |
| Ethyl acetoacetate | 25.62 |
| Dicaprylyl ether | 3.45 |
| 2-Phenylethanol | 3.45 |
| Ethyl cinnamate | 0.42 |
| C13 alcohol ethoxylate (3) | 9.8 |
| Sodium dodecyl sulphate | 4.2 |

### Formulation 6:

| | Content [%] |
|---|---|
| Water | 40 |
| Ethyl acetoacetate | 4 |
| Orange terpene | 36 |
| PEG 7 Glycoyl Cocoate | 16 |
| C9-C11 | 4 |
| alcohol ethoxylate (4) | |

### Formulation 7:

| | Content [%] |
|---|---|
| Water | 38.8 |
| Diacetone alcohol | 2.40 |
| Orange terpene | 38.8 |
| PEG 7 Glycoyl Cocoate | 16 |
| C9-C11 | 4 |
| alcohol ethoxylate (4) | |

### Formulation 8:

| | Content [%] |
|---|---|
| Water | 40 |
| Acetylacetone | 4 |
| Orange terpene | 36 |
| PEG 7 Glycoyl Cocoate | 16 |
| C9-C11 | 4 |
| alcohol ethoxylate (4) | |

### Formulation 9:

| | Content [%] |
|---|---|
| Water | 61.36 |
| NaCl | 0.18 |
| EAA | 13.19 |
| Orange terpene | 13.19 |
| C13 alcohol ethoxylate (8) | 4.59 |
| C13 alcohol ethoxylate (5) | 3.93 |
| Polyoxyethylene(20) sorbitan monostearate | 1.63 |
| Sodium dioctyl sulphosuccinate | 1.93 |

### Formation 10:

| | Content [%] |
|---|---|
| Water | 38.02 |
| Citric acid | 0.56 |
| Arginine | 1.42 |
| Orange terpene | 40 |
| PEG 7 Glycoyl Cocoate | 16 |
| C9-C11 alcohol ethoxylate (4) | 4 |

### Formulation 11:

| | Content [%] |
|---|---|
| Water | 6.43 |
| Ethyl acetoacetate | 13.88 |
| Orange terpene | 57.84 |
| Triethanolamine | 3.43 |
| Oleic acid | 18.42 |

### Formulation 12:

| | Content [%] |
|---|---|
| Water | 46.65 |
| NaCl | 0.43 |
| Glycerol | 0.47 |
| Glycine | 0.35 |
| Ethyl acetoacetate | 12.25 |
| Nicotinamide | 0.31 |
| Ascorbic acid | 0.39 |
| Acetylglycine | 0.50 |
| Dicaprylyl ether | 17.76 |
| Oleyl oleate | 1.99 |
| PEG 7 Glycoyl Cocoate | 11.08 |
| Polyoxyethylene (4) sorbitan monostearate | 7.82 |
| | 100.00 |

### Formulation 13:

| | Content [%] |
|---|---|
| Water | 38.8 |
| NP-MCA | 2.40 |
| Orange terpene | 38.8 |
| PEG 7 Glycoyl Cocoate | 16 |
| C9-C11 | 4 |
| alcohol ethoxylate (4) | |

NP-MCA which can be employed are: dimethylsulphoxide. 2.2.2-trifluoroethanol

### LEGEND TO FIGURES

FIG. 2: a) Essence® ethyl acetate; b) Example 6; c) orange terpenes; d) 2-phenylethanol; e) ethyl acetoacetate

Lackierung . . . =Lacquering with NIVEA® Calcium Power nail varnish

FIG. 10: a) Microemulsion; b) Nanophase fluid

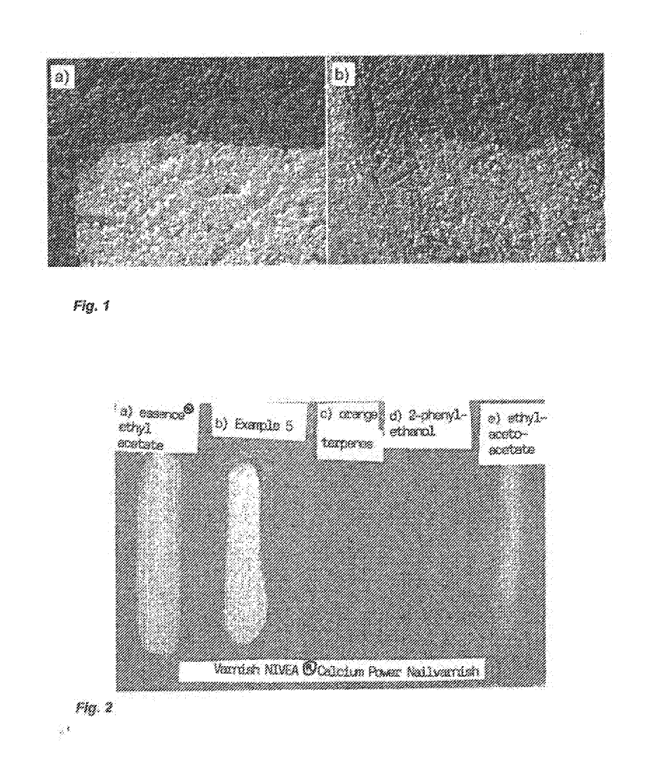

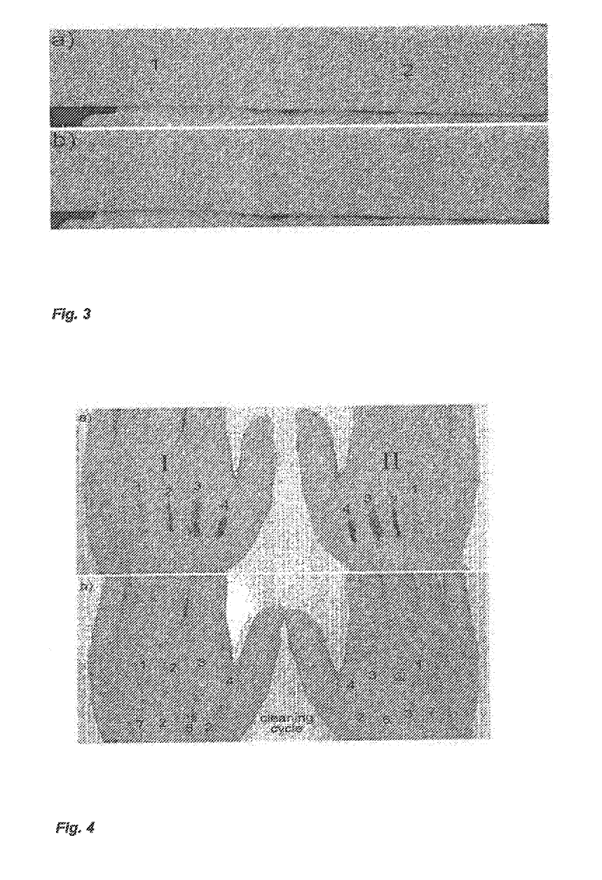

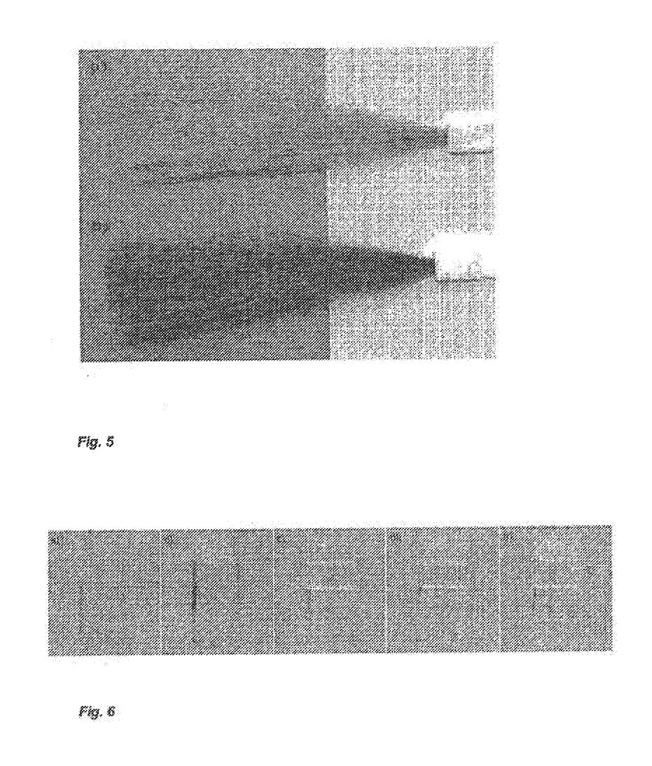

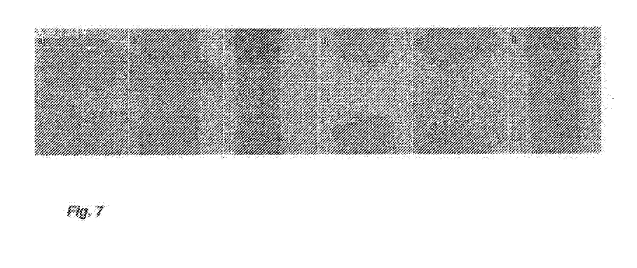

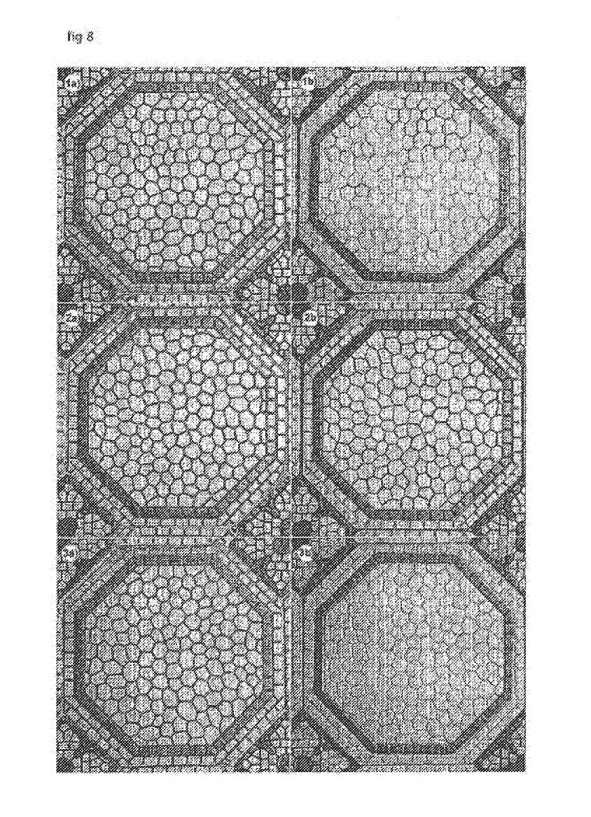

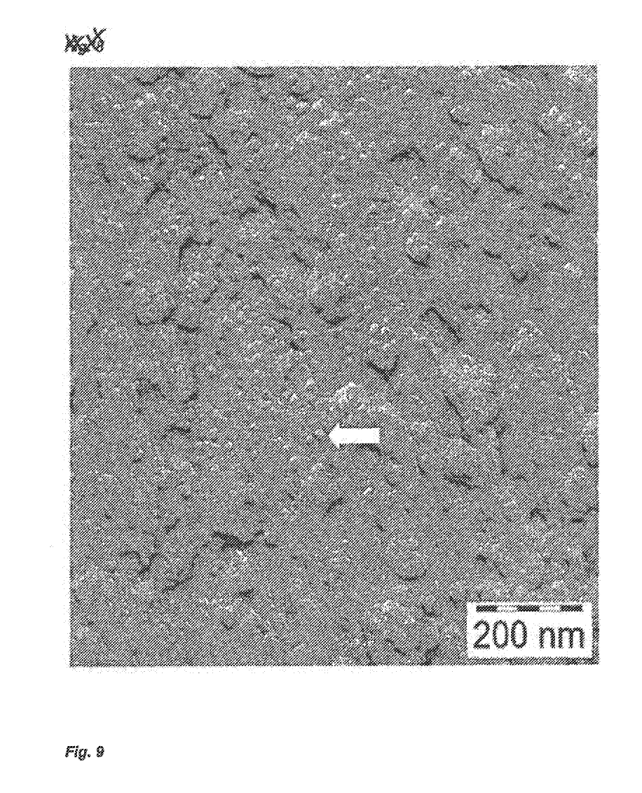

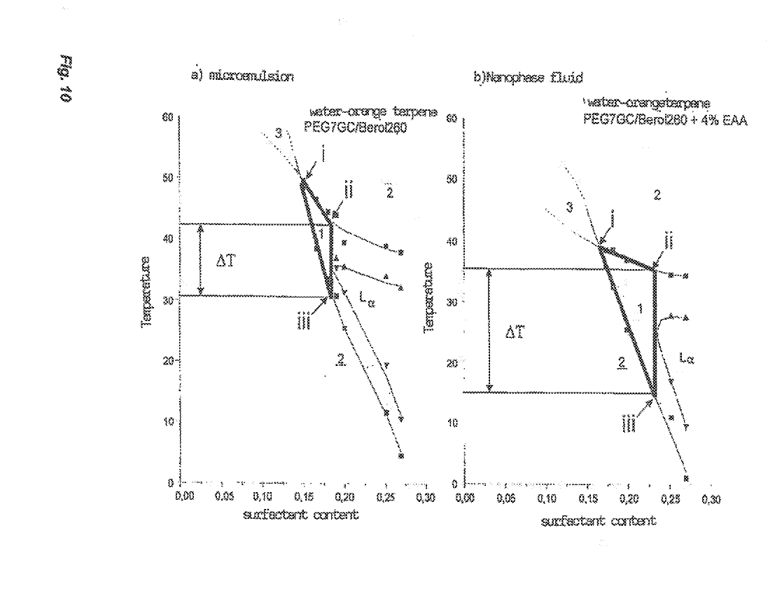

Temperatur=Temperature; Tensidgehalt=Surfactant content; Wasser-Orangenterpen=Water-orange terpene

1. Cleaning composition, characterized in that the cleaning composition contains a microemulsion or a fluid nanophase system and comprises the following constituents:
a) at least one water-insoluble substance with a solubility in water of less than 4 g per litre;
b) at least one amphiphilic substance, NP-MCA, which has no surfactant structure, is not structure-forming by itself, the solubility of which in water or oil is between 4 g and 1,000 g per litre and which does not accumulate preferentially at the oil-water interface, with the proviso that the NP-MCA is not chosen from 2-ethyl-1,3-hexanediol, 2-methyl-2,4-pentanediol, 2-(n-butyl)-2-ethyl-1,3-propanediol and/or from 1,2-diols;
c) at least one anionic, cationic, amphoteric and/or nonionic surfactant;
d) water and/or a water-soluble solvent with hydroxy functionality
and optionally auxiliary substances.

2. Cleaning composition according to claim 1, wherein the NP-MCA is characterized in that on an addition to an oil-water-surfactant system containing the constituents a), c) and d) of 4 wt. %, based on the total weight of the system, it leads to an at least 5% increase in the area of the triangle contained in the phase diagram which is determined by the three corner points:
i) the X point,
ii) the upper point of intersection of the boundary region of the monophase to the two-phase region with the tangent to the start of the Lα region laid parallel to the temperature ordinate and
iii) the lower point of intersection of the boundary region of the monophase to the two-phase region with the tangent to the start of the Lα region laid parallel to the temperature ordinate.

3. Cleaning composition according to claim 1 or 2, characterized in that the cleaning composition comprises a further amphiphilic substance.

4. Cleaning composition according to one of the preceding claims, characterized in that the NP-MCA is chosen from:
a) diols of the formula I:
R₁R₂COH—(CH₂)ₙ—COHR₁R₂ [formula I]
wherein
n can be 0, 1, 2, 3 or 4,
R₁ and R₂ each independently of each other are hydrogen or an unbranched or branched C₁-C₃ alkyl, with the proviso that if n=0, R₁ cannot be hydrogen and the diol is not 2-methyl-2,4-pentanediol;
or
is chosen from 1,3-propanediol, 1,3-butanediol, 1,4-butanediol, 1,5-pentanediol, 1,6-hexanediol, 2,3-butanediol, 2,4-pentanediol or 2,5-dimethyl-2,5-hexanediol,
b) acetoacetates of the formula II:
C(R₃)₃—CO—CH₂—CO—O—R₄ [formula II]
wherein
R₃ each independently of each other is hydrogen or a C₁ to C₂ alkyl and
R₄ is a branched or unbranched C₁ to C₄ alkyl;
or
acetoacetates of the formula III:
CH₃—CO—CH₂—CO—O—R₅ [formula III]
wherein
R₅ is a C₁ to C₄ alkyl;
or is chosen from ethyl acetoacetate, iso-propyl acetoacetate, methyl acetoacetate, n-butyl acetoacetate, n-propyl acetoacetate or tert-butyl acetoacetate,
c) diones of the formula IV
CH₃—(CH₂)ₚ—CO—(CH₂)_{q}—CO—(CH₂)ᵣ—CH₃ [formula IV]
wherein
p, q, r independently of each other can be 0, 1 or 2, with the proviso that if the sum of p, q and r=2, the compound according to formula IV can also be cyclic (cyclohexanedione);
or is chosen from 2,3-butanedione (diacetyl), 2,4-pentanedione (acetylacetone), 3,4-hexanedione, 2,5-hexanedione, 2,3-pentanedione, 2,3-hexanedione, 1,4-cyclohexanedione or 1,3-cyclohexanedione,
d) esters of the formula V
R₆—CO—O—R₇ [formula V]
wherein
R₆ is a ring bond to R₇, CH₃ or COCH₃ and
R₇ is (CH₂)₂—O— ring bond to R₆, (CH₂)₂—O—(CH₂)₃—CH₃, CH₂—CH₃ or CH₂—CH(CH₃)—O— ring bond to R₆;
or is chosen from (1-methoxy-2-propyl)acetate, (2-butoxyethyl)acetate, ethylene carbonate, ethyl pyruvate (2-oxopropionic acid ethyl ester) or propylene carbonate,
e) maleic or fumaric acid amides of the formula VI
R₈—HN—CO—C═C—CO—O—R₉ [formula VI]
wherein
R₈ is hydrogen, a branched or unbranched C₁-C₄ alkyl, or a branched or unbranched, linear or cyclic C₁-C₆ alkyl, wherein the C₁-C₆ alkyl is substituted by one or more groups chosen from OH, NH₂, COOH, CO, SO₃H, OP(OH)₂,
and R₉ is hydrogen or a branched or unbranched C₁-C₄ alkyl;
or is chosen from the following maleic acid amides and methyl, ethyl, propyl and butyl esters thereof: N-methylmaleamide; N-ethylmaleamide; N-(n-propyl)-maleamide; N-(i-propyl)-maleamide; N-(n-butyl)-maleamide; N-(i-butylmaleamide); N-(tert-butylmaleamide), and the corresponding fumaric acid amides and methyl, ethyl, propyl and butyl esters thereof,
f) 2,2-dimethoxypropane, pyruvic aldehyde 1,1-dimethyl acetal, diacetone alcohol (2-methyl-2-pentanol-4-one), 2-butanol, 2-acetyl-gamma-butyrolactone, 3-amino-1H-1,2,4-triazole, gamma-butyrolactone, nicotinamide, ascorbic acid, N-acetylamino acids, in particular N-acetylglycine, -alanine, -cysteine, -valine or -arginine, triethyl phosphate, n-butyl acetate, dimethylsulphoxide or 2,2,2-trifluoroethanol.

5. Cleaning composition according to one of the preceding claims, characterized in that the NP-MCA is chosen from acetoacetates of the formula III
CH₃—CO—CH₂—CO—O—R₅ [formula III]
wherein
R₅ is a C₁ to C₄ alkyl.

6. Cleaning composition according to one of the preceding claims, characterized in that
the water-insoluble substance has a water-solubility of <2 g per litre and the substance is chosen from the group comprising alkanes, cycloalkanes, aromatics, long-chain alkanoic acid esters, esters of di- or tricarboxylic acids, terpenes, or mixtures thereof.

7. Cleaning composition according to one of the preceding claims, characterized in that it has the following composition:
1-90% of water-insoluble substance a)
1-80% of NP-MCA b)
2-45% of surfactant c), including optionally further amphiphilic substance
1-90% of water
optionally auxiliary substances up to a max. of 10%,
the percentage data in each case relating to the total weight of the cleaning composition.

8. Cleaning composition according to one of the preceding claims characterized in that the cleaning composition comprises from 2 to 25 wt. % of NP-MCA, the percentage data relating to the total weight of the cleaning composition.

9. Cleaning composition according to one of the preceding claims, characterized in that the acetoacetate compound is ethyl acetoacetate.

10. Cleaning composition according to one of the preceding claims, characterized in that the cleaning composition comprises from 9 to 16 wt. % of the surfactant according to claim 1c), the percentage data relating to the total weight of the cleaning composition.

11. Process for the preparation of a cleaning composition according to one of claims 1 to 10, characterized in that water or a solvent with hydroxy functionality is initially provided and an anionic, cationic, amphoteric and/or nonionic surfactant is dissolved therein at 10 to 90° C. with stirring, water-insoluble substance(s) are added in parallel with or after the addition of surfactant and the emulsion formed is then converted into a visually transparent microemulsion or a nanophase system by the addition of a further amphiphilic substance and NP-MCA, and auxiliary substances are optionally added at the end of the mixing operation.

12. Method for the removal of undesirable paints and lacquers from surfaces, characterized in that a cleaning composition according to one of claims 1 to 10 is applied to the undesirable paint or the lacquer, acts, and the paint or the lacquer is then removed with water, the action time being from about 10 seconds to about 30 minutes for graffiti removers, from about 20 minutes to about 3 hours for stripping formulations and from about 3 to about 30 seconds for nail varnish removers.

13. Use of the cleaning composition according to claims 1 to 10 as a graffiti remover, stripping formulation or nail varnish remover.

14. Method for the removal of dirt (carbon blacks, fats, oils, silicones, fine dusts, resins and mixtures comprising one or more of these constituents) from surfaces, of cosmetics or for decolorizing hair, characterized in that a cleaning composition according to one of claims 1 to 10 is applied to the dirt, the cosmetics to be removed or the hair to be decolorized, acts, and the dirt, the composition or the colour is then removed with water, the action time being from about 10 seconds to about 3 hours for dirt removers, from about 10 seconds to about 30 minutes for cosmetics removers and from about 2 minutes to about 24 hours for hair decolorizers.

15. Use of the cleaning composition according to claims 1 to 10 as a dirt remover, tile cleaner, cosmetics remover or hair decolorizer.
